(19) United States
(12) Patent Application Publication
(10) Pub. No.: US 2014/0311094 A1
(43) Pub. Date: Oct. 23, 2014

(54) Methods of Increasing the Generation of a Gas Within a Package

(71) Applicant: CRYOVAC, INC., Duncan, SC (US)

(72) Inventors: Patrick W. Thompson, Simpsonville, SC (US); Joshua D. Wofford, Simpsonville, SC (US)

(73) Assignee: CRYOVAC, INC., Duncan, SC (US)

(21) Appl. No.: 14/211,930

(22) Filed: Mar. 14, 2014

Related U.S. Application Data
(60) Provisional application No. 61/812,738, filed on Apr. 17, 2013.

(51) Int. Cl. B65B 55/02; B29D 22/00

(52) U.S. Cl. CPC B65B 55/02; B29D 22/003
USPC 53/425; 156/308.2; 383/116

(57) ABSTRACT

The presently disclosed subject matter relates generally to methods of generating at least one disinfecting gas within a package through a photochemical reaction. More particularly, the disclosed methods include incorporating a composition capable of generating a gas upon exposure to light into the disclosed package. As set forth in detail herein below, the amount of moisture present in the disclosed composition is directly related to the production rate of the gas. Accordingly, the amount of gas generation can be controlled by varying the amount of moisture present in the composition.

### CROSS REFERENCE TO RELATED APPLICATIONS

This application claims the benefit of U.S. Provisional Patent Application No. 61/812,738, filed Apr. 17, 2013, which is incorporated by reference herein in its entirety.

### FIELD OF THE INVENTION

The presently disclosed subject matter relates generally to methods for increasing the rate of generation of at least one gas. More particularly, the presently disclosed subject matter is directed to methods for the increased generation of a sterilizing, sanitizing, or disinfecting gas through optimizing the moisture content of a producing powder.

### BACKGROUND

In the fields of medicine, pharmaceuticals, and food processing there is a critical need for sterilization to protect against the danger of harmful microorganisms. Current industry practices typically employ the use of steam autoclaving to sterilize a wide range of objects. Particularly, steam autoclaving exposes an object to steam at a temperature of about 121° C. at 15-20 lbs per square inch of pressure for 15-30 minutes. The heat and pressure penetrate the object being sterilized and after a sufficient time kill the harmful microorganisms. However, steam autoclaving requires significant user space and resources. Specifically, autoclaving requires cumbersome equipment, a power supply, and extensive plumbing. In addition, steam autoclaving is not suitable for many plastics and other heat-sensitive materials.

Gamma irradiation is also commonly used in the sterilization of medical devices and the like. For example, gamma irradiation involves exposure of an item to a radioactive Cobalt-60 source for a defined amount of time to generate free radicals and other activated molecules that damage the biological components of contaminating bacteria, fungi, and viruses, thereby ensuring their inactivation. The time and exposure to the Cobalt-60 source is used to calculate a total radiation dose for a treated package or pallet, typically ranging from about 50-100 kGy. The required dose for each application is determined based on density, mass, materials, packaging, loading, and the like. However, the free radicals and activated molecules generated as a result of exposure to gamma irradiation have been shown to compromise the mechanical properties of polymers.

Alternatively, sterilant gases (e.g., chlorine dioxide, hydrogen peroxide, and ethylene oxide) can be used to kill or control the growth of microbial contaminants. Chlorine dioxide, for example, is a powerful biocide that can kill fungus, bacteria, and viruses at levels of 0.1 to 10,000 parts per million in contact times of 10 minutes or less. However, one drawback with many of the sterilant gases is that they typically can be used only in limited concentrations and require special handling. In addition, certain sterilants (such as chlorine dioxide, ozone and hydrogen peroxide) are difficult and expensive to transport. Further, many of the sterilant gases must be generated at or near the point of use through on-site plants, which are costly and require significant space to implement.

Thus, there is a need for simple and inexpensive methods and devices for sterilizing objects in a safe and efficient manner. Particularly, it would be desirable to provide methods for the increased chemical reaction rate of a gas.

### SUMMARY

In some embodiments, the presently disclosed subject matter is directed to a method of constructing a package. Particularly, the method comprises providing a polymeric film and a composition that generates a disinfecting gas upon exposure to at least one light source. The disclosed method further comprises either incorporating the composition into the polymeric film during extrusion or coating the composition onto at least one surface of the polymeric film. Further, the method comprises heat sealing the polymeric film to itself or to another film to form an enclosed package for a product. In some embodiments, the disclosed composition comprises about 0.4 to 2.5 weight percent moisture, based on the total weight of the composition.

In some embodiments, the presently disclosed subject matter is directed to a method of disinfecting a product. Specifically, the method includes constructing a package comprising a polymeric film and a composition that generates a disinfecting gas upon exposure to at least one light source incorporated into the polymeric film or coated onto at least one surface of the polymeric film. In some embodiments, the composition comprises about 0.4 to 2.5 weight percent moisture, based on the total weight of the composition. The method further comprises providing the package in an unactivated state, packaging a product within the package, and exposing the package to at least one light source emitting wavelengths between about 350 and 500nm for about 1 to 120 minutes.

In some embodiments, the presently disclosed subject matter is directed to a method of controlling the generation of a disinfecting gas within a package interior. Particularly, the method comprises constructing a package by providing a polymeric film and a composition comprising about 0.4 to 2.5 weight percent moisture, based on the total weight of the composition. The method further comprises either incorporating the composition into the polymeric film during extrusion or coating the composition onto at least one surface of the polymeric film. The method includes heat sealing the polymeric film to itself or to another film to form an enclosed package for a product. The package is provided in an initial unactivated state and is then exposed to at least one light source emitting wavelengths of between about 350 and 500nm for about 1 to 120 minutes. The composition generates a disinfecting gas to disinfect the product wherein the amount of moisture present within said composition is selected in accordance with the amount of disinfecting gas desired.

### BRIEF DESCRIPTION OF THE DRAWINGS

FIG. 1 is a line graph illustrating the upper control limit and percent moisture within three batches of a gas-generating composition as described herein below.

FIG. 2 is a graph illustrating the release profiles of three samples of a gas-generating composition in accordance with some embodiments of the presently disclosed subject matter.

FIG. 3 is a bar graph illustrating the data from FIG. 2.

FIG. 4 is a line graph illustrating the powder moisture content in 7 samples produced according to some embodiments of the presently disclosed subject matter.

### DETAILED DESCRIPTION

### I. General Considerations

The presently disclosed subject matter is generally directed to methods of generating a gas within a package. More particularly, the methods include incorporating a composition capable of generating a gas upon exposure to light into a package. As set forth in detail herein below, it has been determined that the amount of moisture present in the disclosed composition has a significant effect on the production rate of the gas. Thus, the amount of gas generated can be controlled by varying the amount of moisture present in the gas-producing composition.

### II. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long standing patent law convention, the terms “a”, “an”, and “the” refer to “one or more” when used in the subject application, including the claims. Thus, for example, reference to “a package” includes a plurality of such packages, and so forth.

Unless indicated otherwise, all numbers expressing quantities of components, reaction conditions, and so forth used in the specification and claims are to be understood as being modified in all instances by the term “about.” Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, percentage, and the like can encompass variations of, and in some embodiments, ±20%, in some embodiments ±10%, in some embodiments ±5%, in some embodiments ±1%, in some embodiments ±0.5%, and in some embodiments ±0.1%, from the specified amount, as such variations are appropriated in the disclosed films and methods.

The term “disinfecting” or “disinfected” refers to the process of cleansing to destroy and prevent the growth of pathogenic microorganisms. In some embodiments, disinfecting can refer to a combination of a concentration of disinfecting gas and a time exposure interval that will disinfect an object subjected to the gas within a package. Disinfecting conditions can be provided by a wide range of disinfecting gas concentrations in combination with various time intervals. In general, the higher the concentration of a disinfecting gas, the shorter a corresponding time interval needed to establish disinfecting conditions. Accordingly, the effective amount of a disinfecting gas can vary depending upon the length of exposure of the object to the gas. Further, although disinfecting conditions are described herein, in some embodiments the presently disclosed subject matter can equally be used to sterilize a product. Thus, when the term “disinfecting” is used, there are some embodiments where the term includes both disinfecting and sterilizing.

As used herein, the term “disinfecting gas” refers to a gas that effectively destroys, neutralizes, and/or inhibits the growth of pathogenic microorganisms without adversely affecting the object being disinfected. In some embodiments, disinfecting gas includes (but is not limited to) chlorine dioxide, ethylene oxide, sulfur dioxide, nitrogen dioxide, nitric oxide, nitrous oxide, carbon dioxide, hydrogen sulfide, hydrocyanic acid, dichlorine monoxide, ozone, vaporous hydrogen peroxide, and the like. However, this list is not exhaustive and disinfecting gases suitable for use with the presently disclosed subject matter can include any gas that is capable of disinfecting a product.

As used herein, the term “extrusion” is used with reference to the process of forming continuous shapes by forcing a molten plastic material through a die, followed by cooling or chemical hardening. Immediately prior to extrusion through the die, the relatively high-viscosity polymeric material is fed into a rotating screw of variable pitch, which forces it through the die.

As used herein, the term “film” is not limiting and refers to a variety of different flexible materials that can be used in conjunction with the disclosed system including without limitation: a laminate, a web, a sheet, a bag, a pouch, a coating on a substrate or carrier, and the like.

As used herein, the term “heat seal,” and the phrase “heat sealing,” refer to any seal of a first region of a film surface to a second region of a film surface, wherein the seal is formed by heating the regions to at least their respective seal initiation temperatures. The heating can be performed by any one or more known methods, such as using a heated bar, hot wire, hot air, infrared radiation, ultrasonic sealing, etc. In some embodiments, heat sealing is inclusive of thermal sealing, melt-bead sealing, impulse sealing, dielectric sealing, and ultrasonic sealing.

The term “interior” as used herein with regard to a package refers to the actual inside portion of the package into which an object is inserted.

The term “light source” as used herein encompasses all light generating elements and types, including (but not limited to) incandescent, fluorescent, discharge, glow, tungsten halogen, xenon, metal halide, high pressure mercury vapor, arc lamps, sodium vapor lamps, high pressure sodium lamps, induction lamps, electroluminescent, OLED, LED, and the like. The term “light source” as used herein can further refer to a single light source, as well as to a plurality of light sources.

The term “medical product” as used herein refers to any product that is sterilized and/or sanitized prior to use in health care, whether for medical, dental, or veterinary applications. Such products can include (but are not limited to) needles, syringes, sutures, bandages, general wound dressings, non-adherent dressings, burn dressings, surgical tools (such as scalpels, gloves, drapes, and other disposal items), surgical implants, surgical sutures, stents, catheters, vascular grafts, artificial organs, cannulas, wound care devices, dialysis shunts, wound drain tubes, skin sutures, implantable meshes, intraocular devices, heart valves, biological graft materials, tape closures and dressings, head coverings, shoe coverings, sterilization wraps, and the like. Thus, the term “medical product” can include any instrument, apparatus, implement, machine, appliance, contrivance, implant, or other similar or related article, including any component or part that is intended for use in the cure, mitigation, treatment, or prevention of disease, or intended to affect the structure or any function of the body of a human or animal.

The term “moisture” as used herein refers to steam (gas), water (liquid), ice (solid) or any mixture thereof. Thus, in some embodiments, the term “moisture” refers to water.

As used herein, the term “object” refers to the article or material being acted upon to be sterilized, sanitized, disinfected, and/or decontaminated by the disclosed methods and systems. The term can include any material, no matter the physical form. Thus, an object can include, for example, a medical device, medical instrument, or any other article or combination of articles for which disinfection is desired. An object in accordance with the presently disclosed subject matter can have a wide variety of shapes and sizes and can be made from a variety of materials (e.g., without limitation, metal, plastic, glass, and the like).

As used herein, the term “package” can refer to the combination of all of the various components used in the packaging of an object. The package can in some embodiments be inclusive of, for example, a rigid support member, all films used to surround the object and/or the rigid support member, an absorbent component, and even the atmosphere within the package, together with any additional components used in the packaging of the object.

The term “sterilization” as used herein refers to the elimination or destruction of all living microorganisms within a defined system. In the case of a sealed package, for example, the system is defined as the area enclosed by the sealed packaging materials. This area would be the packaged article/device and the headspace.

All compositional percentages used herein are presented on a “by weight” basis, unless designated otherwise.

### III. The Disclosed Package

III.A. Generally

The presently disclosed subject matter relates generally to methods for the generation of a sterilizing gas upon activation of a gas-generating composition. Specifically, the amount of moisture present in the gas-generating composition can be optimized to control the amount of sterilizing gas produced. As set forth herein below, the composition can be incorporated within a package and the package exposed to a light source to generate a desired concentration of gas within the package headspace.

III.B. Gas-Generating Composition

The disclosed gas-generating composition includes an energy-activated catalyst and anions capable of being oxidized by the activated catalyst surface or a subsequent reaction product to generate a gas. To this end, the disclosed composition can comprise from about 50 to 99.9 weight percent energy-activated catalyst; in some embodiments, from about 80 to 98 weight percent energy-activated catalyst; and in some embodiments, from about 86 to 96 weight percent energy-activated catalyst. Thus, the presently disclosed subject matter includes embodiments wherein the composition comprises at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 99.9 weight percent energy-activated catalyst.

Any semiconductor activated by electromagnetic energy, or a particle or other material incorporating such a semiconductor, can be used as the energy-activated catalyst of the composition. Such semiconductors can be metallic, ceramic, inorganic, or polymeric materials prepared by various processes known in the art, such as sintering. In some embodiments, the semiconductors can be surface treated or encapsulated with materials such as silica or alumina to improve durability, dispersibility or other characteristics of the semiconductor. Catalysts for use in the presently disclosed subject matter are commercially available in a wide range of particle sizes from nanoparticles to granules.

In some embodiments, representative energy-activated catalysts can include (but are not limited to) metal oxides (such as anatase, rutile or amorphous titanium dioxide, zinc oxide, tungsten trioxide, ruthenium dioxide, iridium dioxide, tin dioxide, strontium titanate, barium titanate, tantalum oxide, calcium titanate, iron (III) oxide, molybdenum trioxide, niobium pentoxide, indium trioxide, cadmium oxide, hafnium oxide, zirconium oxide, manganese dioxide, copper oxide, vanadium pentoxide, chromium trioxide, yttrium trioxide, silver oxide, and/or TiₓZr₁₋ₓO₂, wherein x is between 0 and 1). In some embodiments, catalysts suitable for use in the presently disclosed subject matter can include (but are not limited to) metal sulfides, such as cadmium sulfide, zinc sulfide, indium sulfide, copper sulfide, tungsten disulfide, bismuth trisulfide, or zinc cadmium disulfide. In some embodiments, presently disclosed catalysts can include (but are not limited to) metal chalcogenites (such as zinc selenide, cadmium selenide, indium selenide, tungsten selenide, or cadmium telluride), metal phosphides (such as indium phosphide), metal arsenides (such as gallium arsenide), nonmetallic semiconductors (such as silicon, silicon carbide, diamond, germanium, germanium dioxide, and germanium telluride), photoactive homopolyanions (such as W₁₀O₃₂⁻⁴), photoactive heteropolyions (such as XM₁₂O₄₀⁻ⁿ or X₂M₁₈O₆₂⁻⁷, wherein x is Bi, Si, Ge, P or As, M is Mo or W, and n is an integer from 1 to 12), and/or polymeric semiconductors (such as polyacetylene).

The disclosed composition comprises from about 0.1 to 50 weight percent anions; in some embodiments, about 2 to 20 weight percent anions; and in some embodiments, from about 4 to 14 weight percent of a source of anions capable of being oxidized by the activated catalyst or reacted with species generated during activation of the catalyst to generate a gas. Therefore, the disclosed composition can comprise no more than about 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 weight percent of a source of anions.

Any source containing anions capable of being oxidized by the activated catalyst or reacted with species generated during excitation of the catalyst to generate a gas can be used in the disclosed composition. An anion is capable of being oxidized by the activated catalyst to generate a gas if its oxidation potential is such that it will transfer an electron to a valence band hole of the energy-activated catalyst. In some embodiments, suitable solids containing anions can include a salt of the anion and a counterion; an inert material such as a sulfate, a zeolite, or a clay impregnated with the anions; a polyelectrolyte such as polyethylene glycol, an ethylene oxide copolymer, or a surfactant; a solid electrolyte or ionomer such as nylon or Nafion™ (DuPont Dow Elastomers (Wilmington, Del., United States of America)); or a solid solution. When the composition is a solids-containing suspension, a salt dissociates in a solvent to form a solution including anions and counterions, and the energy-activated catalyst is suspended in the solution. A powder can be formed, for example, by drying this suspension or by physically blending the solid (e.g., salt particles) with the energy-activated catalyst particles.

Salts suitable for use as the anion source can include (but are not limited to) an alkali metal chlorite, an alkaline-earth metal chlorite, a chlorite salt of a transition metal ion, a protonated primary, secondary or tertiary amine, or a quaternary amine, an alkali metal bisulfite, an alkaline-earth metal bisulfite, a bisulfite salt of a transition metal ion, a protonated primary, secondary or tertiary amine, or a quaternary amine, an alkali metal sulfite, an alkaline-earth metal sulfite, a sulfite salt of a transition metal ion, a protonated primary, secondary or tertiary amine, or a quaternary amine, an alkali metal sulfide, an alkaline-earth metal sulfide, a sulfide salt of a transition metal ion, a protonated primary, secondary or tertiary amine, or a quaternary amine, an alkali metal bicarbonate, an alkaline-earth metal bicarbonate, a bicarbonate salt of a transition metal ion, a protonated primary, secondary or tertiary amine, or a quaternary amine, an alkali metal carbonate, an alkaline-earth metal carbonate, a carbonate salt of a transition metal ion, a protonated primary, secondary or tertiary amine, or a quaternary amine, an alkali metal hydrosulfide, an alkaline-earth metal hydrosulfide, a hydrosulfide salt of a transition metal ion, a protonated primary, secondary or tertiary amine, or a quaternary amine, an alkali metal nitrite, an alkaline-earth metal nitrite, a nitrite salt of a transition metal ion, a protonated primary, secondary or tertiary amine, or a quaternary amine, an alkali metal hypochlorite, an alkaline-earth metal hypochlorite, a hypochlorite salt of a transition metal ion, a protonated primary, secondary or tertiary amine, or a quaternary amine, an alkali metal cyanide, an alkaline-earth metal cyanide, a cyanide salt of a transition metal ion, a protonated primary, secondary or tertiary amine, or a quaternary amine, an alkali metal peroxide, an alkaline-earth metal peroxide, or a peroxide salt of a transition metal ion, a protonated primary, secondary or tertiary amine, or a quaternary amine. In some embodiments, preferred salts can include sodium, potassium, calcium, lithium or ammonium salts of a chlorite, bisulfite, sulfite, sulfide, hydrosulfide, bicarbonate, carbonate, hypochlorite, nitrite, cyanide or peroxide. Commercially available forms of chlorite and other salts suitable for use, can contain additional salts and additives such as tin compounds to catalyze conversion to a gas.

The sterilizing or sanitizing gas released by the disclosed composition will depend upon the anions that are oxidized or reacted. Any gas formed by the loss of an electron from an anion, by reaction of an anion with electromagnetic energy-generated protic species, by reduction of a cation in an oxidation/reduction reaction, or by reaction of an anion with a chemisorbed molecular oxygen, oxide or hydroxyl radical can be generated and released by the composition. For example, in some embodiments, the gas can be chlorine dioxide, sulfur dioxide, hydrogen sulfide, hydrocyanic acid, nitrogen dioxide, nitric oxide, nitrous oxide, carbon dioxide, dichlorine monoxide, chlorine, and/or ozone.

In some instances, the disclosed composition can include two or more different anions to release two or more different gases at different rates. The gases can be released for different purposes or so that one gas will enhance the effect of the other gas. For example, a composition containing bisulfite and chlorite anions can release sulfur dioxide for food preservation and chlorine dioxide for control of microorganisms.

In some embodiments, the disclosed composition can be a solid (such as a powder), depending upon its intended use. For example, when a powder is exposed to electromagnetic energy, the energy-activated catalyst is activated, the anions are oxidized or reacted with species generated during excitation of the catalyst to generate the gas, and the gas diffuses through the powder and is released. However, the disclosed composition is not limited solely to a powder and can be embodied in any physical form.

The disclosed composition can comprise any of a wide variety of additives known in the art. Such additives can include (but are not limited to) colorants, dyes, fragrances, fillers, lubricants, stabilizers, accelerators, retarders, enhancers, blending facilitators, controlled release agents, antioxidants, UV blockers, mold release agents, plasticizers, biocides, flow agents, anti-caking agents, processing aids, and/or light filtering agents. Continuing, additives such as UV blockers can also be included in the disclosed composition if it is desirable to limit the wavelength range transmitted to the energy-activated catalyst. Photosensitizers can further be added to shift the absorption wavelength of the composition, particularly to shift an ultraviolet absorption wavelength to a visible absorption wavelength to improve activation by room lighting. UV absorbers can be added to the composition to slow the gas generation and release rate.

III.C. Package

Applications for the disclosed gas-generating composition are numerous. For example, the composition can be incorporated into a package for disinfecting the package contents. Particularly, the composition can be impregnated, melt processed, sintered, blended with other powders, or otherwise incorporated into a variety of materials to provide films, fibers, coatings, tablets, resins, polymers, plastics, tubing, membranes, engineered materials, paints and adhesives for a wide range of end use applications. For example, the disclosed composition can in some embodiments be incorporated into injection-molded, compression-molded, thermal-formed and/or extrusion-formed polymeric products by compounding and/or pelletizing the disclosed composition via conventional means and mixing the pellets with a material before the conventional forming or molding process. Thus, in some embodiments, the composition can be useful in preparing a wide variety of injection-molded products, compression-molded products, thermal-formed products, and/or extrusion-formed products, such as cast or blown films.

For instance, in some embodiments a multilayered film can be used to construct a package that can generate a gas within the package interior. In such embodiments, at least one package film can include a gas generating layer comprising the disclosed composition. Particularly, the gas-generating layer can comprise an energy-activated catalyst capable of being activated by electromagnetic energy and anions capable of being oxidized or reacted to generate a gas. In some embodiments, the disclosed package can comprise a barrier layer positioned adjacent to a surface of the gas generating layer. The barrier layer can in some embodiments be transparent to electromagnetic energy such that it transmits the energy to the gas generating layer. The barrier layer can be impermeable or only semipermeable to the gases generated and released by the gas generating layer.

Therefore, the surface of a material (such as a polymeric film) or the entire material itself can be impregnated or coated with the disclosed composition. Alternatively or in addition, the composition can be admixed with a material, the composition can be enclosed within a gas-permeable container, and/or the material and the composition can be enclosed within a container. When the composition is enclosed within a container, the container can be hermetically or partially sealed such that some gas leaks from the container.

Products (such as packages, films, and the like) can include between about 0.1 and 70 weight percent of the disclosed composition; in some embodiments, from about 1 to 50 weight percent of the disclosed composition; and in some embodiments, from about 2 to 50 weight percent of the disclosed composition. Thus, the composition can be present in a product in an amount of at least or no more than about 0.1, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 weight percent. As would be appreciated by those of ordinary skill in the art, the remainder of the products can be comprised of polymeric materials, additives, and the like.

III.D. Product

The disclosed package can be used to house any of a wide variety of products known in the art. For example, in some embodiments, the disclosed package can be used to house medical products, food products, various industrial and consumer products, and other like objects.

### IV. The Disclosed Methods

The presently disclosed subject matter includes a method for optimizing the release of a gas to sterilize and/or disinfect objects, such as for medical applications. It has been surprisingly discovered that when the amount of moisture present in the disclosed composition is about 0.4 to 2.5 weight % (based on the total weight of the composition), the amount of sterilizing gas generated is about 0.75 to 2.00 ppm/mg composition. Thus, in some embodiments, the disclosed method comprises providing the disclosed composition with a moisture content of about 0.4 to 2.5 weight percent; in some embodiments, about 0.5 to 2.4 weight percent; in some embodiments, about 0.75 to about 2.2 weight percent; and in some embodiments, about 1.0 to about 2.0 weight percent, based on the total weight of the composition. In some embodiments, the optimal moisture content of the disclosed powder is about 2.0 weight percent, based on the total weight of the composition.

In some embodiments, the disclosed composition can be prepared by combining the individual components (energy-activated catalyst, anions, additives) in a fluid (such as water) to make a suspension. Once the suspension is formed, it can be spray dried to form a powder by any method known in the art. For example, any known atomization methods such as nozzles or rotary discs can be used. Thus, in some embodiments, the spray drying process begins with atomization of a liquid feed into a spray of fine droplets. The spray can then be contacted with (and suspended by) a heated gas stream, allowing the liquid to evaporate and leave the dried solids in essentially the same size and shape as the atomized droplet. Finally, the dried powder can be then separated from the gas stream and collected. The spent drying gas can be treated to meet environmental requirements and then exhausted to the atmosphere or, in some cases, recirculated to the system. In some embodiments, the spray drying process can occur rapidly (e.g., within up to about 60 seconds). If desired, the powder can then be further dried by any conventional method. After production, the powders are stored under conditions where they are not exposed to electromagnetic energy of a wavelength that would activate the catalyst (e.g., in dark conditions for photoactive catalysts).

The spray drying conditions can be controlled to ensure that the powder is produced with a moisture content of about 0.4 to 2.5 weight %. For example, in some embodiments, the outlet gas temperature, inlet gas temperature, fuel supply rate, quench air volume, slurry feed rate, transportation air, and/or slurry solids content can be controlled or modified.

For example, the slurry feed rate is the rate at which the suspension is fed into the spray drying apparatus. In some embodiments, to optimize the moisture content of the powder, the slurry feed rate can be about 70 to 300 pounds per hour. However, as would be known to those of ordinary skill in the art, the slurry feed rate used to produce the disclosed composition can include a wide range of rates and is not limited to the range set forth above.

The inlet temperature is the temperature of the air entering the spray drying apparatus. Similarly, the outlet temperature is the temperature of the air exiting the spray drying apparatus. The moisture content of the disclosed composition can be optimized by controlling the inlet and/or outlet temperature. Specifically, in some embodiments, the inlet and outlet temperatures are maintained at about 800° F.-1400° F. and about 150° F.-220° F., respectively. However, these temperatures can vary as would be apparent to those of skill in the art.

The quench air volume is the volume of the gas stream that enters the spray drying apparatus. In some embodiments, the moisture content of the disclosed composition can be controlled by optimizing the quench air volume. Particularly, in some embodiments, the quench air volume can be 500 to 2000 standard cubic feet per minute. However, the quench air volume used in the presently disclosed subject matter can vary widely, as would be apparent to those of skill in the art.

The solids content is defined as the mass of all solids dissolved or suspended in a slurry divided by the total mass of the slurry, including the liquid or water component. In the disclosed applications, the solids content is the percent of the slurry available for collection as product from the spray drying process, which can be in powder form. Suitable solids contents can range from about 15% to about 40% by mass, although the percentage is not limited to the range set forth herein.

The fuel supply rate is defined as the volume of fuel supplied to the spray dryer combustor over time. In some embodiments, fuel is supplied to the spray dryer to feed a combustion reaction of fuel and supply/combustion air. The hot gases produced by the reaction are fed to the top of the spray drying chamber and used to dry the injected or atomized slurry feed. Typical fuel supply rates can be able 5 to 10 standard cubic feet per minute, although greater or lesser rates are also included within the scope of the presently disclosed subject matter.

The transportation air is defined as the volume of gas stream that is used to convey a powder after it is spray dried. In some embodiments, transportation air can be used to pneumatically transport powder from one location to another. In some embodiments, transportation air can also be used to influence the moisture content of a powder. In particular, when a powder is warmer than about 100° F., it can (in some embodiments) be dried beyond what is achieved in the actual spray drier. Transportation air can also be conditioned to introduce moisture to the surface of the powder if needed.

Thus, in some embodiments, the outlet gas temperature, inlet gas temperature, fuel supply rate, quench air volume, slurry feed rate, transportation air, and/or slurry solids content can be controlled to ensure that the generated powder includes a moisture content of about 0.4 to 2.5 weight %, based on the total weight of the powder.

The produced powder can then be incorporated or coated onto or into a package as set forth in more detail herein above. When the disclosed package is exposed to a light source, the composition generates a disinfecting gas. Without being bound by any particular theory, it is believed that the energy-activated catalyst of the composition absorbs a photon having energy in excess of the band gap and an electron is promoted from the valence band to the conduction band, producing a valence band hole. The valence band hole and electron diffuse to the surface of the energy-activated catalyst where each can chemically react. An anion is oxidized by the activated catalyst surface when an electron is transferred from the anion to a valence band hole, forming the gas. It is believed that chlorine dioxide or nitrogen dioxide is generated by the transfer of an electron from a chlorite or nitrite anion to a valance band hole. These and other gases, such as ozone, chlorine, carbon dioxide, nitric oxide, sulfur dioxide, nitrous oxide, hydrogen sulfide, hydrocyanic acid, and dichlorine monoxide, can also be formed via reaction of an anion with protic species generated during activation of the catalyst by abstraction of an electron from water, chemisorbed hydroxyl, or some other hydrated species. See, U.S. Pat. No. 7,273,567, the entire disclosure of which is hereby incorporated by reference.

The disinfecting gas diffuses out of the composition and into the surrounding atmosphere (such as the interior of a package) for a period of up to about 24 hours. In some embodiments, the gas can be used to retard, control, kill and/or prevent microbiological contamination (e.g., bacteria, fungi, viruses, mold spores, algae, and protozoa). Alternatively or in addition, the gas can be used to deodorize, enhance freshness, and/or retard, prevent, inhibit, and/or control chemotaxis. In some embodiments, the concentration of disinfecting gas within the package is about 75 to 300 ppm for a duration of 10 minutes to 24 hours.

It should be recognized that the methods listed herein above are not intended to be limiting. Rather, the presently disclosed subject matter includes the methods described above, in addition to any of a wide variety of methods known in the art.

### V. Advantages of the Presently Disclosed Subject Matter

The presently disclosed subject matter is directed to methods of controlling the generation of a desired gas within a package. Particularly, the method includes the step of optimizing the moisture content of the disclosed composition to about 0.4 to 2.5 weight percent, based on the total weight of the composition. As a result, the disclosed gas-generating composition exhibits increased production of the disinfecting gas.

Further, by optimizing the conditions needed to retard, prevent or control biological contamination, disinfection time is reduced, thereby allowing more efficient time to market.

In addition, the ability to increase the sterilizing gas concentration at a desired level facilitates a more controlled and robust microbial inactivation or sterilization system.

Although several advantages of the disclosed system are set forth in detail herein, the list is by no means limiting. Particularly, one of ordinary skill in the art would recognize that there can be several advantages to the disclosed system and methods that are not included herein.

### EXAMPLES

The following Examples provide illustrative embodiments. In light of the present disclosure and the general level of skill in the art, those of ordinary skill in the art will appreciate that the following Examples are intended to be exemplary only and that numerous changes, modifications, and alterations can be employed without departing from the scope of the presently disclosed subject matter.

### Example 1

### Preparation of Samples 1-3

Sample 1 was prepared by spray dying a pre-mixed slurry composed of 26 weight % solids (897.2 lbs) and 74 weight % water (2553.9 lbs). The solids component was formulated with 77 weight percent titanium dioxide, 10 weight percent sodium chlorite, 5 weight percent sodium carbonate, 3 weight percent calcium stearate, 3 weight percent sodium dodecyl sulfate, and 2 weight percent sodium hydroxide. Thus, 690.9 lbs titanium dioxide, 44.9 lbs sodium carbonate, 26.9 lbs calcium stearate, 26.9 lbs sodium dodecyl sulfate, and 17.9 lbs sodium hydroxide were added to 2426.2 lbs of water and mixed with a high shear mixer (Fristam Mixing Table, Model 10-52, available from Fristam Pumps USA, Middleton, Wis., United States of America) for about 2.5 hours. The slurry was then mixed for 10-14 hours with an impeller mixer. The slurry was completed by adding 89.2 lbs sodium chlorite and 127 lbs water. The slurry was mixed with the high shear mixer for an additional 1 hour. The finished slurry was then pumped to a feed tank and spray dried to produce Sample 1 according to the following conditions: contact temperature 945° F., exit temperature 210.2° F., average pump speed (feed rate) of 16.0%, wet feed solids 26.7%, and dry product moisture 0.38%.

Sample 2 was prepared by aging Sample 1 in a dark room at room temperature for about 2 months.

Sample 3 was prepared by aging Sample 1 for about 2 months under the same conditions as used for Sample 2, and then storing the sample for 4 hours at 80% relative humidity.

### Example 2

Moisture Content Testing of Sample 1

About 5 g of Sample 1 was added to a 50 mL clear glass vial. The vial was immediately wrapped in an opaque material for testing. A standard aluminum tray was tared on a moisture content analyzer (Denver Instruments IR-200, available from Denver Instrument, Bohemia, N.Y., United States of America). 2 g of Sample 1 was removed from the vial and placed on the tared tray. The sample was heated to 110° C. for about 6 minutes and the moisture content was measured 5× by the moisture content analyzer to have 0.38% moisture. The test is a loss in weight function (i.e., when the sample stops losing weight, it is considered dry and the test stops).

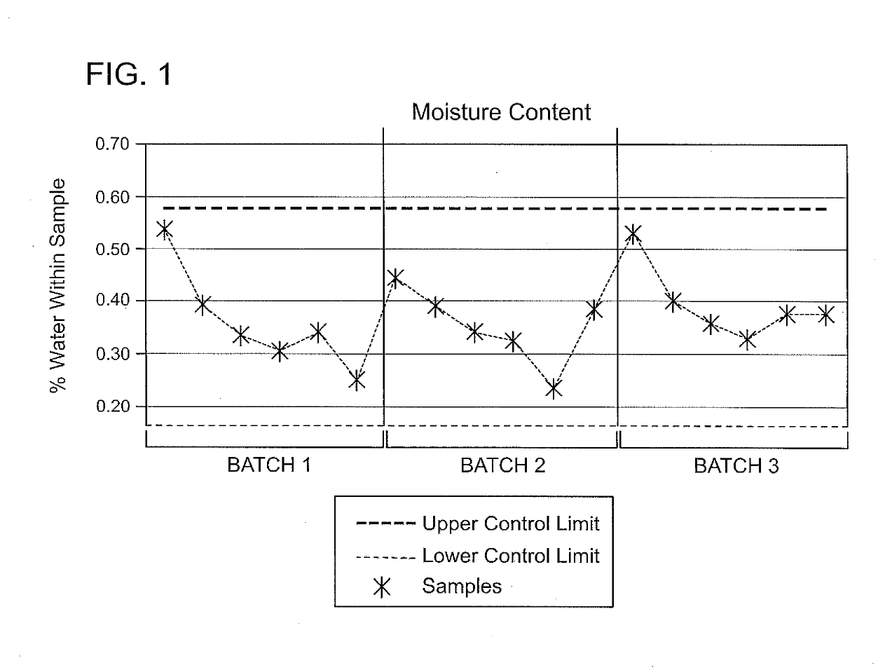

The percent water within Sample 1 is given below in Table 1 and is represented graphically in FIG. 1.

TABLE 1
Moisture Content Testing of Sample 1

| | Trial 1 | Trial 2 | Trial 3 | Trial 4 | Trial 5 | |
|---|---|---|---|---|---|---|
| Sample | (%) | (%) | (%) | (%) | (%) | Avg (%) |
| 1 | 0.33 | 0.40 | 0.35 | 0.34 | 0.46 | 0.38 |

### Example 3

### Activity Testing of Samples 1-3

Four electrochemical cells (ECs, identified as A, B, C, D) (7CLH CiTiceL, available from City Technology, Ltd., Hampshire, United Kingdom; Serial Nos. 853167012, 2378092043, 2378211043, and 2378046043) were attached to the lid of an inverted clear jar so that the membrane of the ECs were exposed to the closed atmosphere within the jar and the electrical circuitry of each EC was attached to the exterior of the jar. The electrical circuitry was used to amplify the electrical response of the EC to mVs. A small metal mesh stand was used to support a circular paper disc for holding Sample 1. A fluorescent ballast with two fluorescent bulbs was attached to the lid of a metal box that surrounded the EC array, thereby creating a dark environment with the lights off and a lit environment from a single source with the lights on. The circuitry of the EC cells was connected to a TA-Instruments DAQ (TA Instruments, New Castle, Del., United States of America) that relayed the data to a computer for recording.

A pre-weighed 1⅝ inch diameter circular piece of filter paper was used to measure the amount of Sample 1 used (under yellow light). The sample was then reweighed to determine the amount of sample tested, as set forth in Table 2 below. The filter paper and sample were then placed onto the metal mesh stand and attached to the inverted lid of the clear glass jar. The jar was then tightened onto the lid, sealing the sample inside the jar interior. The metal box containing fluorescent ballasts was then placed on top of the EC cells, thus surrounding the EC array.

The fluorescent light was turned on and the amount of chlorine dioxide generated by each sample was measured at 5 and 120 minute timepoints, as set forth below in Table 3. Each sample was run in quadruplicate, at the same time, on four different EC cells. However, the data from EC B for Sample 1 was omitted after the instrument did not pass calibration.

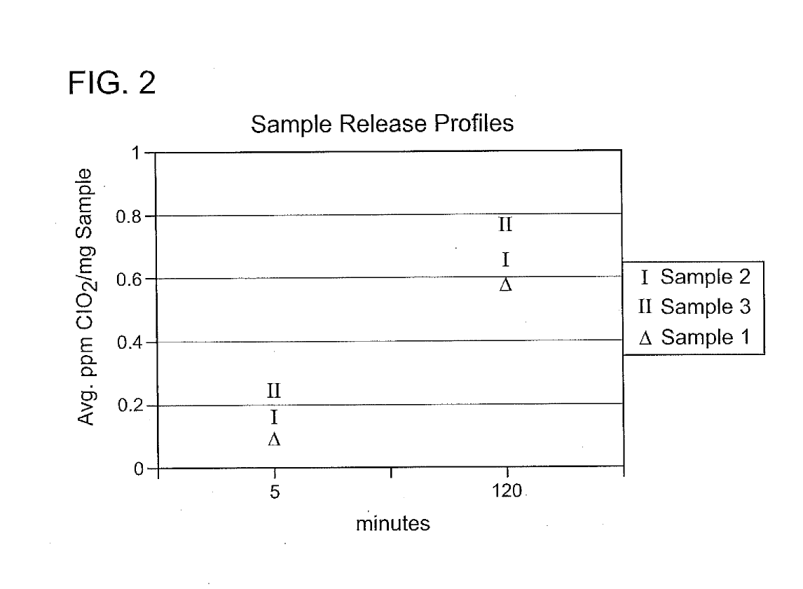

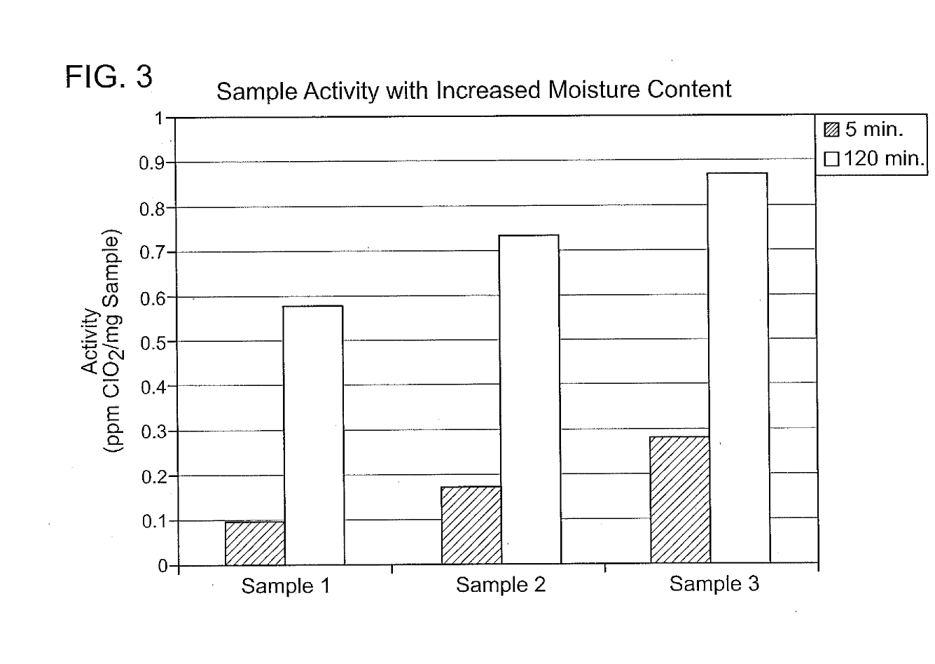

Samples 2 and 3 were tested using the sample procedure described above for Sample 1. FIG. 2 is a graph illustrating the average release profile of chlorine dioxide for Samples 1-3 at 5 and 120 minute timepoints, taken from the data in Tables 2-3 below. This data is also graphically represented in FIG. 3 as a bar graph.

TABLE 2
Mass Measurements for Samples 1-3

Mass Measurements

| | | | | Weight |
|---|---|---|---|---|
| | | Filter | Paper + | Post |
| | EC | paper | Sample | Experiment |
| Sample | CELL | (g) | (g) | (g) |
| 1 | A | 0.181 | 0.211 | 0.211 |
| | B | 0.182 | 0.211 | 0.211 |
| | C | 0.183 | 0.213 | 0.213 |
| | D | 0.185 | 0.217 | 0.218 |
| 2 | A | 0.181 | 0.197 | 0.197 |
| | B | 0.184 | 0.203 | 0.203 |
| | C | 0.183 | 0.207 | 0.207 |
| | D | 0.183 | 0.202 | 0.203 |
| 3 | A | 0.175 | 0.197 | 0.197 |
| | B | 0.174 | 0.190 | 0.190 |
| | C | 0.174 | 0.197 | 0.196 |
| | D | 0.177 | 0.198 | 0.196 |

TABLE 3
Activity Measurements for Samples 1-3

| | Activity Measurements | | | | |
|---|---|---|---|---|---|
| Sample | DAQ Channel | A | B | C | D |
| 1 | Array Position | 1 | —* | 6 | 7 |
| | Amount Sample 1 | 30 | —* | 30 | 32 |
| | (mg) | | | | |
| | ppm ClO₂ at t = 5 | 2.8 | —* | 2.9 | 2.7 |
| | min. | | | | |
| | ppm ClO₂/mg | 0.095 | —* | 0.097 | 0.085 |
| | Sample 1 at t = 5 | | —* | | |
| | min | | | | |
| | ppm ClO₂ at | 18.3 | —* | 17.9 | 17.4 |
| | t = 120 min | | | | |
| | ppm ClO₂/mg | 0.611 | —* | 0.596 | 0.544 |
| | Sample 1 at t = 5 | | | | |
| | min | | | | |
| 2 | Array Position | 1 | 2 | 6 | 7 |
| | Amount Sample 1 | 16 | 19 | 24 | 19 |
| | (mg) | | | | |
| | ppm ClO₂ at t = 5 | 2.2 | 4.1 | 3.9 | 3.2 |
| | min. | | | | |
| | ppm ClO₂/mg | 0.139 | 0.213 | 0.162 | 0.170 |
| | Sample 1 at t = 5 | | | | |
| | min | | | | |
| | ppm ClO₂ at | 9.2 | 18.2 | 15.8 | 14.2 |
| | t = 120 min | | | | |
| | ppm ClO₂/mg | 0.573 | 0.960 | 0.660 | 0.748 |
| | Sample 1 at t = 5 | | | | |
| | min | | | | |
| 3 | Array Position | 1 | 2 | 6 | 7 |
| | Amount Sample 1 | 22 | 16 | 23 | 21 |
| | (mg) | | | | |
| | ppm ClO₂ at t = 5 | 5.4 | 6.0 | 6.0 | 4.8 |
| | min. | | | | |
| | ppm ClO₂/mg | 0.244 | 0.373 | 0.260 | 0.230 |
| | Sample 1 at t = 5 | | | | |
| | min | | | | |
| | ppm ClO₂ at | 20.1 | 18.5 | 17.2 | 13.6 |
| | t = 120 min | | | | |
| | ppm ClO₂/mg | 0.915 | 1.155 | 0.749 | 0.648 |
| | Sample 1 at t = 5 | | | | |
| | min | | | | |

*There was a system error in Activity Cell B for Sample 1, so the data was omitted.

### Conclusions from Examples 1-3

Examples 1-3 illustrate that the activity level of the powder increased with increasing moisture content (%) over the range of about 0.4 to 2.5 wt % moisture. Specifically, Sample 1 is considered to be the low moisture content analog with an approximate moisture content of 0.4% and an activity level of about 0.6 ppm ClO₂ per mg of sample (as illustrated in FIG. 3).

Sample 2 can be considered the medium moisture content analog. Moisture content testing was not conducted at the time activity testing was done for Samples 2 and 3. However, the powder has a hygroscopic nature and would be expected to increase in moisture content over time during a “natural” aging process under the conditions performed in Examples 1-3. The activity level of Sample 2 was about 0.75 ppm ClO₂ per mg of sample (as shown in FIG. 3).

Sample 3 is considered the high moisture content analog. Sample 3 was created by exposing additional Sample 2 material to a very humid environment to actively increase the sample moisture content. The activity level of Sample 3 was about 0.85 ppm ClO₂ per mg sample (as shown in FIG. 3).

### Example 4

### Preparation of Samples 4-10

Samples 4-10 were prepared using the method set forth in Example 1, except the contact temperature was 1244° F., the exit temperature was 168° F., the average pump speed (feed rate) was 46.8%, the wet fee solids content was 26.4%, and the dry moisture content was variable. A total of 7 samples (identified as Samples 4-10) were collected.

### Example 5

### Moisture Content Testing of Samples 4-10

Samples 4-10 were placed in lidded glass jars, covered with aluminum foil, and stored in dark conditions for 3 months prior to testing. The particle size range for Samples 4-10 was 43-52 microns, average d(0.9). The particle size distribution was achieved after several days of process experimentation. The moisture content was tested using the method set forth in Example 2. The results are shown in Table 4 below.

TABLE 4
Moisture Content of Samples 4-10

| | Trial 1 | Trial 2 | Trial 3 | | | |
|---|---|---|---|---|---|---|
| Sample | (% | (% | (% | Trial 4 (% | Trial 5 (% | Avg. (% |
| ID | water) | water) | water) | water) | water) | water) |
| 4 | 1.19 | 1.12 | 1.09 | 1.05 | 1.27 | 1.14 |
| 5 | 0.97 | 0.89 | 0.89 | 0.96 | 1.00 | 0.94 |
| 6 | 0.85 | 0.95 | 0.97 | 1.02 | 0.91 | 0.94 |
| 7 | 0.98 | 0.95 | 0.85 | 0.90 | 0.79 | 0.89 |
| 8 | 0.65 | 0.66 | 0.78 | 0.70 | 0.79 | 0.72 |
| 9 | 0.69 | 0.94 | 0.79 | 0.77 | 0.84 | 0.81 |
| 10 | 0.61 | 1.08 | 0.82 | 0.90 | 0.78 | 0.84 |

### Example 6

### Activity Testing of Samples 4-10

4 electrochemical cells (ECs, identified as A, B, C, D, available from City Technology Ltd., Hampshire, United Kingdom, Serial Nos. 23181343, 23181344, 23181348, and 23181356) were attached through the rear of 4 polystyrene dessicator cabinets (Model 2618100, available from Hatch Co., Loveland, Colo., United States of America) such that the membrane of the ECs were exposed to the closed atmosphere within each cabinet and the electric circuitry of each EC was attached to the exterior of each cabinet. The electrical circuitry was used to amplify the electrical response of the EC to mAs. The enclosed volume of each cabinet was about 4.9 liters. A plastic sample dish was used to hold each sample. An ultraviolet light emitting diode (LED) assembly (available from LED Wholesalers, Hayward, Calif., United States of America, Item No. 1328UV395) was mounted directly above each cabinet, thereby creating a dark environment with the lights off and a lit environment from a source for each cabinet with the lights on. The circuitry of the EC cells was connected to an Advantech Data Acquisition Module (ADAM 4017+ and ADAM-4520-D2E) that relayed the data to a computer for recording.

4 identical custom 3D printed sample dishes were used to measure the amount of powder of each sample and to provide a uniform surface area of powder for activation. The diameter of each dish was 35 mm and the depth was 3 mm. Each empty dish was weighed and loaded with the amount of sample to be tested. Each powder sample was leveled across the top of the dish using a glass slide. The sample and dish were then reweighed for a final mass and loaded into the cabinet for activation. Cabinets were loaded with one dish per cabinet and then closed to create a sealed area.

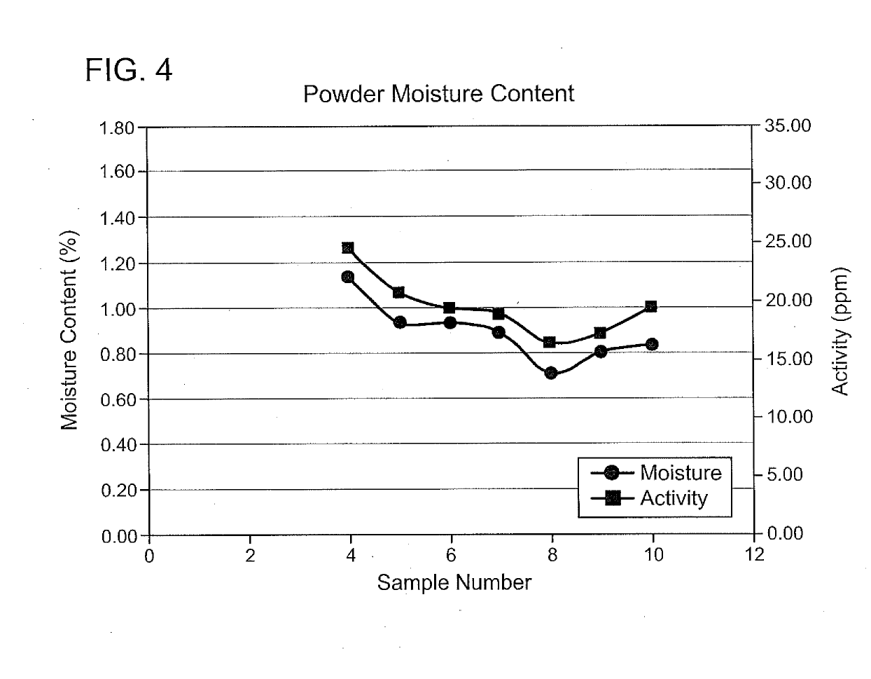

The UV LED was then turned on and the amount of chlorine dioxide generated by each sample was measured over a period of about 45 minutes. The maximum concentration of gas was recorded for each sample. Each sample was run 4× at the same time on 4 different EC cells. The average of the 4 different EC cells for each sample is shown below in Table 5. The relationship between the moisture content and activity for Samples 4-10 is illustrated in FIG. 4 (prepared from the data given in Tables 4 and 5).

TABLE 5
Activity Testing of Samples 4-10

| Sample ID | Avg (%) |
|---|---|
| 4 | 24.56 |
| 5 | 20.82 |
| 6 | 19.53 |
| 7 | 19.00 |
| 8 | 16.50 |
| 9 | 17.34 |
| 10 | 19.49 |

### Conclusions from Examples 4-6

Examples 4-6 illustrate that increasing the moisture content of the powder increases the activity level. Without being bound by any particular theory, it is believed that the increased moisture at the surface of the photocatalyst increases the reaction kinetics and thus increases the release of gas (activity) from the surface of the spray dried particle. Thus, the powder can deliver a greater amount of disinfecting gas from a fixed mass of powder by elevating the moisture available at activation.

For the purposes of downstream processing associated with polymeric film or masterbatch processing (i.e., extrusion), the maximum moisture content should be less than about 2.5% to ensure that downstream processing is conducted without issues such as foaming, cavitation, or degradation in high temperature processing.

What is claimed is:

1. A method of constructing a package, said method comprising:
a. providing a polymeric film;
b. providing a composition that generates a disinfecting gas upon exposure to at least one light source;
c. either:
i. incorporating said composition into the polymeric film during extrusion; or
ii. coating said composition onto at least one surface of the polymeric film;
d. heat sealing said polymeric film to itself or to another film to form an enclosed package for a product,
wherein said composition comprises about 0.4 to 2.5 weight percent moisture, based on the total weight of the composition.

2. The method of claim 1, wherein said disinfecting gas is selected from the group consisting of: chlorine dioxide, ethylene oxide, sulfur dioxide, hydrogen sulfide, hydrocyanic acid, nitrogen dioxide, nitric oxide, nitrous oxide, carbon dioxide, vaporous hydrogen peroxide, dichlorine monoxide, chlorine, ozone, or combinations thereof.

3. The method of claim 1, wherein said composition comprises an energy-activated catalyst and anions capable of being oxidized by the activated catalyst or a subsequent reaction product to generate a gas.

4. The method of claim 3, wherein the catalyst is selected from the group comprising: metal oxides, metal sulfides, and metal chalcogenites.

5. The method of claim 3, wherein said composition comprises two or more different anions.

6. The method of claim 1, wherein said product is a medical product.

7. The package produced by the method of claim 1.

8. A method of disinfecting a product, said method comprising:
a. constructing a package comprising:
i. a polymeric film;
ii. a composition that generates a disinfecting gas upon exposure to at least one light source incorporated into said polymeric film or coated onto at least one surface of said polymeric film, said composition comprising about 0.4 to 2.5 weight percent moisture, based on the total weight of the composition;
b. providing said package in an unactivated state;
c. packaging said product within said package;
d. exposing said package to at least one light source emitting wavelengths between about 350 and 500 nm for about 1 to 120 minutes;
wherein said composition generates a disinfecting gas at a concentration of about 0.75 to 200 ppm/mg to disinfect said product.

9. The method of claim 8, wherein said disinfecting gas is selected from the group consisting of: chlorine dioxide, ethylene oxide, sulfur dioxide, hydrogen sulfide, hydrocyanic acid, nitrogen dioxide, nitric oxide, nitrous oxide, carbon dioxide, vaporous hydrogen peroxide, dichlorine monoxide, chlorine, ozone, or combinations thereof.

10. The method of claim 8 wherein said composition comprises an energy-activated catalyst and anions capable of being oxidized by the activated catalyst or a subsequent reaction product to generate a gas.

11. The method of claim 10, wherein the catalyst is selected from the group comprising: metal oxides, metal sulfides, and metal chalcogenites.

12. The method of claim 10, wherein said composition comprises two or more different anions.

13. The method of claim 8, wherein said product is a medical product.

14. The package disinfected by the method of claim 8.

15. A method of controlling the generation of a disinfecting gas within a package interior, said method comprising:
a. constructing a package by:
i. providing a polymeric film;
ii. providing a composition comprising about 0.4 to 2.5 weight percent moisture, based on the total weight of the composition,
iii. either:
1. incorporating said composition into the polymeric film during extrusion; or
2. coating said composition onto at least one surface of the polymeric film;
iv. heat sealing said polymeric film to itself or to another film to form an enclosed package for a product,
b. providing said package in an unactivated state;
c. exposing said package to at least one light source emitting wavelengths of between about 350 and 500nm for about 1 to 120 minutes;
wherein said composition generates a disinfecting gas to disinfect said product and wherein the amount of moisture present within said composition is selected in accordance with the amount of disinfecting gas desired.

16. The method of claim 15, wherein said disinfecting gas is selected from the group consisting of: chlorine dioxide, ethylene oxide, sulfur dioxide, hydrogen sulfide, hydrocyanic acid, nitrogen dioxide, nitric oxide, nitrous oxide, carbon dioxide, vaporous hydrogen peroxide, dichlorine monoxide, chlorine, ozone, or combinations thereof.

17. The method of claim 15, wherein said composition comprises an energy-activated catalyst and anions capable of being oxidized by the activated catalyst or a subsequent reaction product to generate a gas.

18. The method of claim 17, wherein the catalyst is selected from the group comprising: metal oxides, metal sulfides, and metal chalcogenites.

19. The method of claim 17, wherein said composition comprises two or more different anions.

20. The method of claim 15, wherein said product is a medical product.
